(19) United States
(12) Patent Application Publication
(10) Pub. No.: US 2017/0184472 A1
(43) Pub. Date: Jun. 29, 2017

(54) SENSOR ARRANGEMENT AND MEASUREMENT METHOD FOR A TURBOMACHINE

(71) Applicant: Rolls Royce Deutschland Ltd & Co KG, Blankenfelde-Mahlow (DE)

(72) Inventors: Stefan FECHNER, Berlin (DE); Stefan ALBELT, Berlin (DE)

(21) Appl. No.: 15/380,389

(22) Filed: Dec. 15, 2016

(30) Foreign Application Priority Data
Dec. 24, 2015 (DE) 10 2015 226 732.6

(51) Int. Cl. G01M 15/14; G01N 33/00

(52) U.S. Cl. CPC G01M 15/14; G01N 33/0004

(57) ABSTRACT

A sensor arrangement with a sensor element for measuring at least one physical and/or chemical fluid characteristic in a turbomachine is provided. The sensor element detects the at least one fluid characteristic inside a non-contact seal, in particular a labyrinth seal, between a rotor stage and a stator stage, wherein during operation the sensor element is in contact with the fluid flow along the flow path inside the labyrinth seal.

### REFERENCE TO RELATED APPLICATION

This application claims priority to German Patent Application No. 10 2015 226 732.6 filed on Dec. 24, 2015, the entirety of which is incorporated by reference herein.

### BACKGROUND

The invention relates to a sensor device for a turbomachine and a measurement method.

In turbomachines, in particular in aircraft engines, fluid flows play a role in many areas. Thus, it is for example necessary to use cooling air and to monitor its temperature in different areas of the turbomachine. A sudden temperature rise of the cooling air can be an indication of a problem, or it may itself cause a problem. It may also be necessary to monitor working fluids, such as for example oil flows.

### SUMMARY

Therefore, there is the objective to create sensor arrangements and measurement methods which facilitate an efficient and reliable monitoring of fluids inside the turbomachine.

At that, the sensor element detects at least one fluid characteristic inside a non-contact seal between a rotor stage and a stator stage, wherein during operation the sensor element is in (direct or indirect) contact with the fluid flow along the flow path inside the non-contact seal. Thus, the detection takes place directly inside the seal and not in an upstream or downstream position.

In one embodiment, a pressure gradient is present across the flow path, so that a fluid is transported in the non-contact seal, in particular the labyrinth seal.

In a further embodiment, the rotor stage and the stator stage are arranged inside a turbine, in particular a high-pressure turbine, wherein the seal with the sensor element is located near the gas path, in order to provide a seal against the entry of hot gases and if necessary to detect the entry of a hot gas.

Here, the sensor element can be arranged at the beginning, in the middle or at the end of the flow path through the non-contact seal, in particular the labyrinth seal. In addition or as an alternative, the sensor element can also be arranged inside a measuring chamber, which is connected through a channel with the actual measuring point in the seal, and thus the fluid to be detected first has to be guided to the sensor element through a channel.

When the sensor arrangement is applied, the sensor element can measure a temperature of the fluid flow, in particular of an air flow, a mass flow of the fluid flow, in particular an oil flow, and/or a composition of the fluid flow, in particular of a combustion gas. For this purpose, for example optical sensors can be used that can detect the composition, the conductivity or the optical permeability of the fluid flow.

Here, it is also possible that the sensor element is coupled to a control device 52 by means of which a control signal can be emitted based on the measurement of the sensor element 51, which may for example inform the pilot in the cockpit about the measurement, or may initiate an automatic switch-off of the aircraft engine.

The objective is also achieved through a measurement method with features as described herein.

### BRIEF DESCRIPTION OF THE DRAWINGS

Embodiments of the invention are shown in an exemplary manner based on the following figures.

FIG. 1 shows a schematic rendering of an aircraft engine.

FIG. 2 shows a schematic simplified sectional view of a first exemplary embodiment of the sensor arrangement according to the invention in an axial sectional plane.

FIG. 3 shows a schematic simplified sectional view of a second exemplary embodiment of the sensor arrangement according to the invention in an axial sectional plane.

FIG. 4 shows a schematic view of a case of error as hot gas is sucked in through a labyrinth seal.

FIG. 5 shows a schematic view of a case of error where an oil leak occurs in a non-contact seal.

FIG. 6 shows a schematic view of a case of error during combustion.

FIG. 7 shows a perspective rendering of a real non-contact seal with an embodiment of a sensor arrangement.

### DETAILED DESCRIPTION

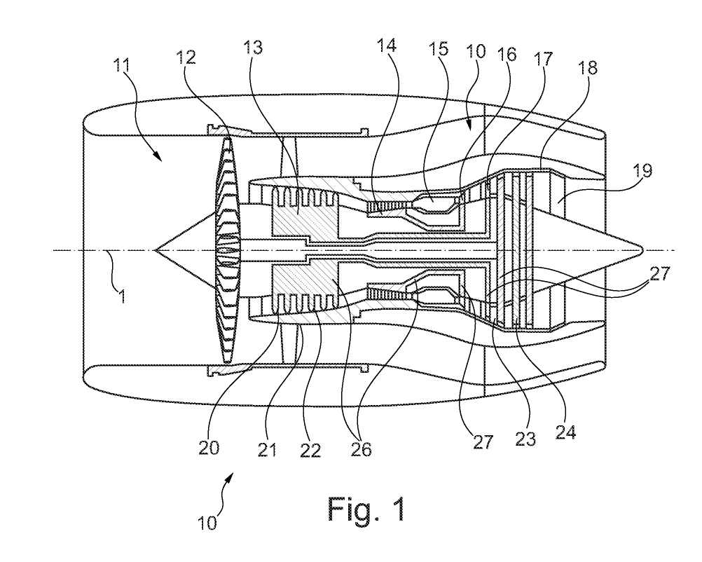

The aircraft engine 10 according to FIG. 1 shows a general example of a turbomachine.

In most cases, the aircraft engine 10 is embodied in a per se known manner as a multi-shaft engine and comprises, arranged in succession in flow direction, an air inlet 11, a fan 12 rotating inside a housing, where applicable a medium-pressure compressor 13, a high-pressure compressor 14, a combustion chamber 15, a high-pressure turbine 16, where applicable a medium-pressure turbine 17 and a low-pressure turbine 18, as well as an exhaust nozzle 19, which all are arranged around a central engine axis 1.

The medium-pressure compressor 13 and the high-pressure compressor 14 respectively comprise multiple stages, of which each has an arrangement of fixedly attached stationary guide vanes 20 extending in the circumferential direction, which are generally referred to as stator vanes and which protrude radially inwards from the engine shroud 21 through the compressors 13, 14 into a ring-shaped flow channel. Further, the compressors have an arrangement of compressor rotor blades 22, which protrude radially outwards from a rotatable drum or disc 26 and which are coupled to turbine rotor hubs 27 of the high-pressure turbine 16 or of the medium-pressure turbine 17.

The turbines 16, 17, 18 have similar stages, comprising an arrangement of stationary guide vanes 23, which protrude radially inwards from the housing 21 through the turbines 16, 17, 18 into the ring-shaped flow channel, and a subsequent arrangement of turbine blades 24 which protrude outwards from the rotatable turbine rotor hub 27. During operation, the compressor drum or compressor disc 26 and the blades 22 arranged thereon as well as the turbine rotor hub 27 and the turbine blades 24 arranged thereon rotate around the engine central axis 1.

Often there is a gap between the stators and the rotors of the compressors 13, 14 and the turbines 16, 17, 18 directly at the flow channel or also further inside the engine, through which fluids, such as for example cooling air or oil, may leak. These gaps are usually provided with non-contact seals 40, such as for example a labyrinth seal. Here, it is important for the functionality of the aircraft engine 10 that the fluid flow through the seal 40 is monitored.

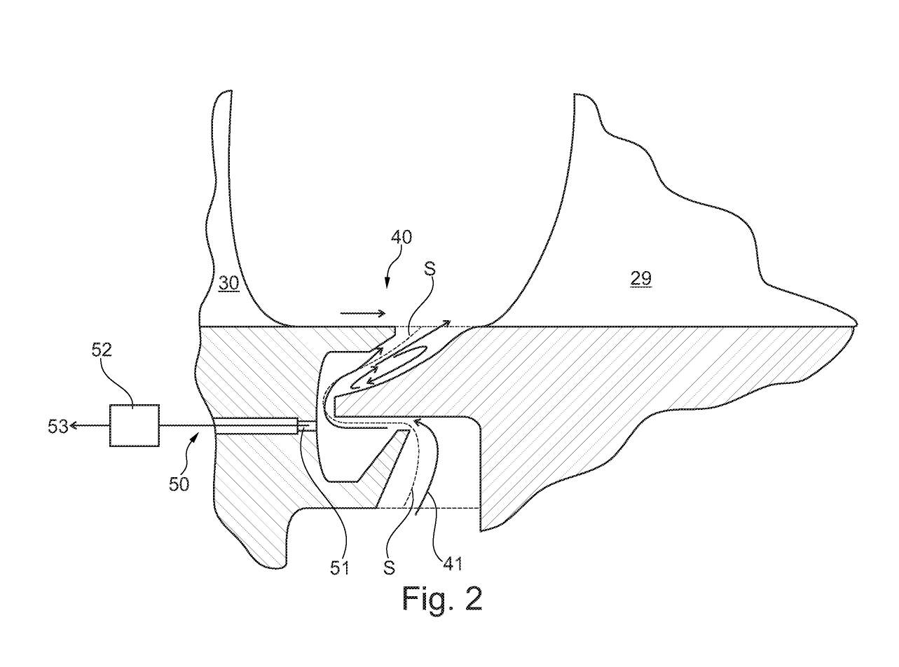

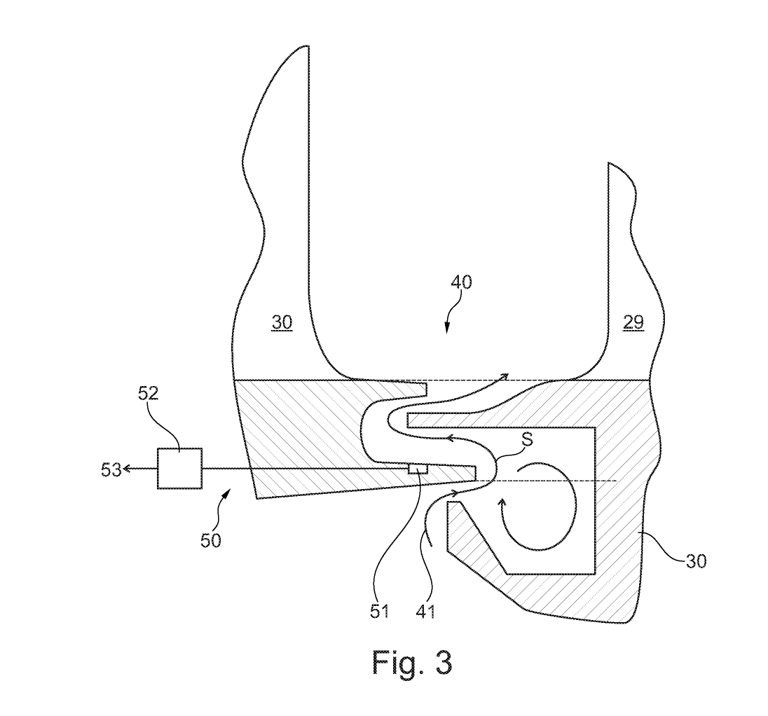

FIGS. 2 and 3 show embodiments of a labyrinth seal 40 with a sensor arrangement 50 for fluid monitoring. In a labyrinth seal 40, the sealing effect is based on an elongation of the flow path inside the gap to be sealed, whereby the flow resistance is increased. Typically, a labyrinth seal 40 has meshing parts (combing).

The sectional planes of FIGS. 2 and 3 comprise the engine axis 1 and thus extend in the radial direction.

FIGS. 2 and 3 respectively show an exemplary embodiment in which the flow direction extends from left to right in the aircraft engine 10. Thus, the turbine flow first flows through a stator stage 30 with guide vane leafs before impinging on a rotor stage 29 with rotor blade leafs.

In FIG. 2, the labyrinth seal 40 is arranged between the stator disc 30 and the rotor disc 29. The fluid flow 41—in this case air, for example—flows radially form inside out along the flow path S. At that, the flow path is delimited radially inwards and outwards by the dashed line.

Here, a sensor element 51 is arranged directly at the flow path S as a part of a sensor device 50, by means of which a fluid characteristic, namely the temperature inside the labyrinth seal 40, is measured. In the process, the sensor element 51 is in (direct or indirect) contact with the fluid flow 41 along the flow path S inside the labyrinth seal 40. In the present case, the sensor element 41 is arranged approximately in the middle of the flow path S, and is oriented in such a manner that the fluid flow 41 flows towards the sensor element 41.

In FIG. 3, an alternative sensor arrangement 50 inside a labyrinth seal 40 is shown, wherein the description of FIG. 2 may be referred to. Here, too, the sensor element 51 is arranged approximately in the middle of the flow path S. But in this case, the flow impinges on the sensor element 51 tangentially.

In alternative embodiments, the sensor element 41 is arranged at the beginning or at the end of the flow path S. It is also possible for the sensor element 51 to be arranged not at a right angle to the flow path S, but at an acute or obtuse angle. In any case, it has direct contact with the fluid flowing inside the labyrinth seal 40.

In the embodiments of FIGS. 2 and 3, the sensor element 51 of the sensor arrangement 50 is connected to a control device 52 that can emit a control signal 53 if for example a certain measurement value—here a temperature value—is exceeded or undershot.

If, for example due to a malfunction, hot gas would flow from the outside into the labyrinth seal 40, the temperature rise would be detected. This could then be translated into a control signal 53 by the control device 52.

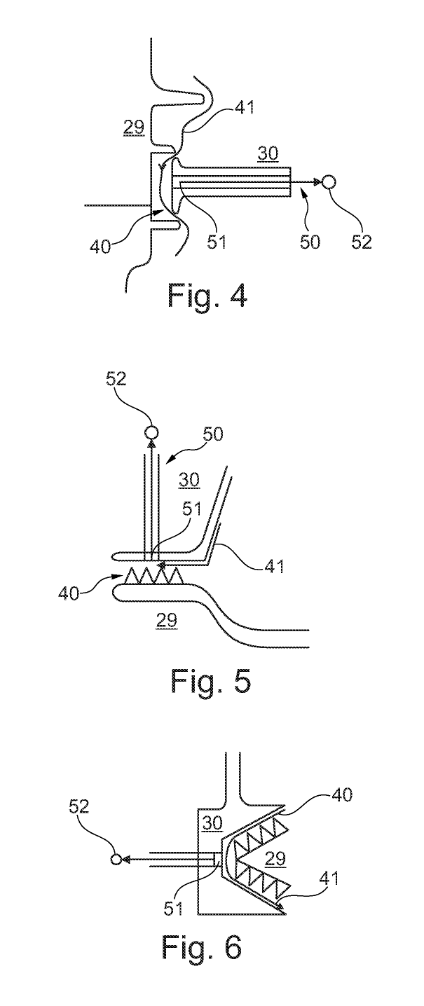

In FIGS. 4 to 6, some such cases are shown in a schematic manner. In all three cases, the sensor element 51 is arranged approximately in the middle of the fluid flow 41. The sensor arrangement is respectively arranged in the stator stage 30.

FIG. 4 shows a labyrinth seal 40 that is arranged between a rotor stage 29 to the left and a stator stage 30 to the right. What is shown here is a case in which hot gas flows from the outside radially inward through the labyrinth seal 40 in the event of a damage to the aircraft engine 10. The sensor element 51 detects the temperature rise that is an indicator for the damage and passes the information on to the control device 52. If a defined threshold value is exceeded, the pilot in the cockpit could be informed by a signal. Another possibility would be an automatic switch-off of the aircraft engine.

FIG. 5 shows a further application of the sensor arrangement 50 which is designed for the detection of oil leaks. Here, a non-contact seal 40 is arranged between a stator stage 30 above and a rotor stage 29 that is located below the same. A sensor element 51, for example of an optical oil sensor, for example detects a leak of a bearing chamber in the event that the oil flow exceeds a certain amount. The measurement result is forwarded to the control device 52.

In FIG. 6, a labyrinth seal 40 is arranged between a stator stage 30 to the left and a rotor stage 29 to the right. Here, an optical sensor detects whether combustion products and/or solid bodies flow through the labyrinth seal 40 together with the fluid. In this case, the optical characteristics (for example the extinction) of the fluid would change, so that a case of error due to the entry of combustion gases via the seal element could be detected. Also in this case, the measurement result is passed on to the control device 52.

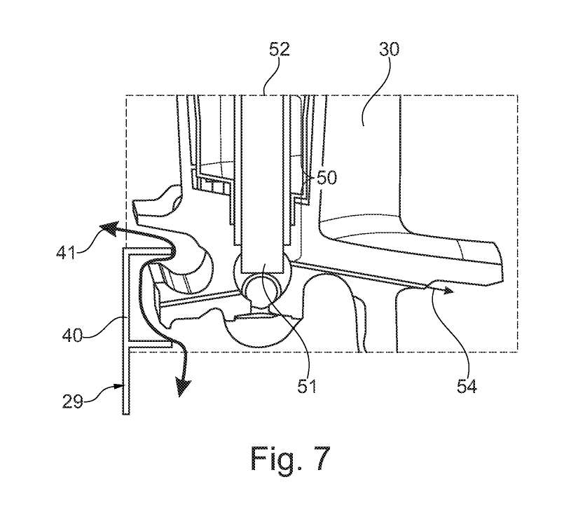

FIG. 7 shows a perspective view of a non-contact seal 40 between a stator stage and a rotor stage of a turbine. FIG. 7 shows the fluid flow 41 that occurs due to an unplanned suctioning in of a hot gas for example. In this case, the sensor element 51 does not operate directly at the site of the fluid flow 41. However, in order to also be able to immediately detect the hot gas before it advances further into the aircraft engine 10, the fluid flow 41 is suctioned through a pressure gradient that is present above the stator stage, and is guided past the sensor element via the air channel 54. By designing a short channel, a delayed response to the entry of the hot gas is minimized, but also a simplified design, here in particular of a straight sensor element 51, is facilitated. This simple constructional design of the sensor facilitates an exchange of the element with engines that are still mounted at the aircraft.

In the embodiment shown herein, the sensor arrangement 50 has an approximately cylindrical sensor element 51.

### Parts list

1 engine axis
10 gas turbine engine, aircraft engine
11 air inlet
12 fan
13 medium-pressure compressor (compactor)
14 high-pressure compressor
15 combustion chamber
16 high-pressure turbine
17 medium-pressure turbine
18 low-pressure turbine
19 exhaust nozzle
20 compressor guide vanes
21 engine shroud
22 compressor rotor blades
23 turbine guide vanes
24 turbine rotor blades
26 compressor drum or compressor disc
27 turbine rotor hub
29 rotor stage
30 stator stage
40 seal, labyrinth seal
41 fluid flow
50 sensor arrangement
51 sensor element
52 control device
53 control signal
54 air channel
S flow path inside the labyrinth seal

1. A sensor arrangement with a sensor element for measuring at least one physical and/or chemical fluid characteristic in a turbomachine, wherein the sensor element detects at least one fluid characteristic in a non-contact seal, in particular a labyrinth seal, between a rotor stage and a stator stage, wherein during operation the sensor element is in contact with the fluid flow along the flow path in the non-contact seal.

2. The sensor arrangement according to claim 1, wherein a pressure gradient is present across the flow path, so that a fluid is transported through the non-contact seal, in particular the labyrinth seal.

3. The sensor arrangement according to claim 1, wherein the rotor stage and the stator stage are arranged inside a turbine, in particular in a high-pressure turbine, wherein the seal with the sensor element is positioned close to the gas path, in order to provide sealing against the entry of a hot gas and to detect the entry of a hot gas, if necessary.

4. The sensor arrangement according to claim 1, wherein the sensor element is arranged at the beginning, in the middle or at the end of the flow path through the non-contact seal, in particular the labyrinth seal, and/or that the sensor element is positioned inside a measuring chamber which is connected to the actual measuring point inside the seal through a channel, and thus the fluid to be detected first has to be guided to the sensor element through a channel.

5. The sensor arrangement according to claim 1, wherein a temperature of the fluid flow, in particular of an air flow, a mass flow of the fluid flow, in particular of an oil flow, and/or a composition of the fluid flow, in particular of a combustion gas, can be measured by means of the sensor element.

6. The sensor arrangement according to claim 1, wherein the sensor element is coupled to a control device by means of which a control signal can be emitted based on the measurement with the sensor element.

7. A measurement method with a sensor element for measuring at least one physical and/or chemical fluid characteristic in a turbomachine, wherein the sensor element detects the at least one fluid characteristic in a non-contact seal, in particular a labyrinth seal, between a rotor stage and a stator stage, wherein the measurement is carried out by the sensor element during operation through contact with the flow of the fluid along the flow path in the non-contact seal.
